(19) United States
(12) Patent Application Publication
(10) Pub. No.: US 2026/0027251 A1
(43) Pub. Date: Jan. 29, 2026

(54) BASE STATIONS HAVING OCCUPANCY SENSORS, DOOR MOVEMENT SENSORS, AND/OR AIR DISINFECTION CAPABILITY IN CONJUCTION WITH PORTABLE AREA/ROOM DISINFECTION APPARATUSES

(71) Applicant: XENEX DISINFECTION SERVICES INC., San Antonio, TX (US)

(72) Inventors: Tuck Stibich, Santa Fe, NM (US); Juan Gonzalez, San Antonio, TX (US); Deepak Jayaraj, Pittsburgh, PA (US)

(21) Appl. No.: 19/343,009

(22) Filed: Sep. 29, 2025

Related U.S. Application Data
(63) Continuation of application No. PCT/US2024/021720, filed on Mar. 27, 2024.
(60) Provisional application No. 63/456,267, filed on Mar. 31, 2023.

(51) Int. Cl. A61L 2/26; A61L 2/10; A61L 2/24

(52) U.S. Cl. CPC A61L 2/26; A61L 2/10; A61L 2/24; A61L 2202/11; A61L 2202/14; A61L 2202/16; A61L 2202/25

(57) ABSTRACT

Receptacles and systems having occupancy sensors and/or air disinfection capability in conjunction with portable area/room disinfection apparatuses are provided. An embodiment of a receptacle for receiving a portable disinfection apparatus includes a sensor for detecting occupancy or potential occupancy in an area/room and a wireless transmitter communicably coupled to the sensor. Embodiments of other receptacles include an air moving device for drawing air from an ambient of receptacle and configurations to provide an air channel from the air moving device to an area at which germicide from a portable disinfection apparatus is emitted when the portable disinfection apparatus is arranged in the receptacle. An embodiment of a system includes a receptacle for receiving a portable disinfection apparatus and an air duct fluidly coupled to a port of the receptacle and extending through a wall of a room in which the receptacle is arranged.

### PRIORITY CLAIM

This application is a continuation application of International Application No. PCT/US2024/021720 filed Mar. 27, 2024, which designates the United States and claims priority to U.S. Provisional Patent Application No. 63/456,267 filed Mar. 31, 2023.

### BACKGROUND OF THE INVENTION

### 1. Field of the Invention

This invention generally relates to base stations for portable disinfection apparatuses and, more specifically, receptacles having occupancy sensors, door movement sensors, and/or air disinfection capability in conjunction with portable area/room disinfection apparatuses.

### 2. Description of the Related Art

The following descriptions and examples are not admitted to be prior art by virtue of their inclusion within this section.

Surface disinfection in rooms and areas is becoming increasingly important as antimicrobial resistant organisms are becoming more prevalent and increasingly difficult to treat. In general, the objective of a surface disinfection process performed in an area or room is to reduce the number of pathogenic microorganisms in the air and/or on surfaces in the area/room to a level which is much less harmful to human health. To limit or prevent exposure of germicides and/or distractions to occupants of a room or area, area/room surface disinfection is typically performed by trained cleaning personnel or by an automated device which disperses a germicide into an ambient of a room after the room has been vacated by the previous occupants. As an added benefit, automated area/room disinfection apparatuses innately disinfect some of the air in the area or room by the dispersion of the germicide from the device to the surfaces. It is often desirable, however, to conduct air disinfection processes in occupied rooms without exposing individuals to germicides. Considering this, separate air and surface disinfection systems are often utilized in a single room.

Although all-in-one devices exist which can conduct automated surface disinfection when an area/room is unoccupied and also conduct air surface disinfection in the area/room when it is occupied, such devices typically require air moving devices, filters, and actuators to move components of the systems to provide selectivity between the air and surface disinfection options. Such features undesirably increase the cost of the devices as well as the complexity of programming the devices. Furthermore, the features increase power requirements of the devices, which may deter from maximizing the disinfection efficiency of the devices (i.e., may deter from maximizing the amount and/or distribution of germicide from the unit in a given amount of time). Moreover, the noted features may increase weight of the devices, which may be particularly undesirable for portable devices.

As noted above, automated area/room disinfection apparatuses are often used in vacated areas/rooms in order to limit or prevent exposure of germicides to individuals. In order to ensure an area/room has been vacated and continues to be unoccupied for a disinfection cycle, automated area/room disinfection apparatuses often include occupancy sensors to monitor the area/room for the presence of human or animals and are programmed to inhibit or terminate the distribution of a germicide if occupancy is detected. Having such occupancy sensors on area/room disinfection robots that are configured for autonomous movement is particularly important since such robots are typically configured to move to different areas/rooms of a building, making them suspectable to encounter people or animals in a facility. Due to their automated movement, however, motion sensors are not typically a viable option to Use as occupancy sensors for such devices. Consequently, other types of occupancy sensors must be used on disinfection apparatuses configured for automated movement, such as but not limited to thermal sensors, Doppler sensors, or photo recognition sensors, all of which are generally known to be more costly and/or more complicated to use than motion sensors.

Accordingly, it would be beneficial to develop portable area/room surface disinfection apparatuses and systems which are cheaper and less complex than conventional devices, but safe to use in an occupied facility. In addition, it would also be beneficial to develop cheaper portable area/room disinfection apparatuses and systems having less complexity, but which enable surface disinfection when an area/room is unoccupied and air surface disinfection when the area/room is occupied.

### SUMMARY OF THE INVENTION

The following description of various embodiments of receptacles and systems is not to be construed in any way as limiting the subject matter of the appended claims.

An embodiment of a receptacle for storing a portable disinfection apparatus includes a bay for receiving the portable disinfection apparatus and a first sensor for detecting a parameter indicative of occupancy of a human or an animal in a room or area in which the receptacle is arranged or potential occupancy of a human or animal occupancy in the room or area. In addition, the receptacle includes a wireless transmitter communicably coupled to the first sensor for sending a signal into an ambient of the room upon the first sensor detecting the parameter.

An embodiment of a system includes a portable disinfection apparatus and a receptacle for the portable disinfection apparatus, wherein the receptacle comprises a first sensor comprising a human or animal occupancy sensor or a door movement sensor. The receptacle further includes a wireless transmitter communicably coupled to the first sensor for sending a signal to inhibit or terminate operation of the portable disinfection apparatus upon the first sensor detecting the parameter.

An embodiment of another system includes a portable disinfection apparatus having a germicidal source, an air inlet along its exterior surface, an air outlet along its exterior surface, and an air passage between the air inlet and air outlet that includes an area internal to the portable disinfection apparatus to which the germicidal source is configured to emit a germicide. The system further includes a receptacle for the portable disinfection apparatus, wherein the receptacle includes an air moving device for drawing air from an ambient of the receptacle and a bay for receiving the portable disinfection apparatus. The receptacle is configured to provide an air channel from the air moving device to the air inlet of the portable disinfection apparatus when the portable disinfection apparatus is arranged in the bay.

An embodiment of another system includes a portable disinfection apparatus comprising a germicidal source arranged to emit a germicide to an area external to the portable disinfection apparatus and a receptacle for the portable disinfection apparatus. The receptacle includes an air moving device for drawing air from an ambient of the receptacle and a bay for receiving the portable disinfection apparatus. The receptacle is configured to enclose the portable disinfection apparatus in the bay and provide an air channel from the air moving device to an ambient of the portable disinfection apparatus when the portable disinfection apparatus is arranged in the bay.

An embodiment of another system includes a receptacle for housing a portable disinfection apparatus, wherein the receptacle comprises a bay for receiving the portable disinfection apparatus and a port along a surface of the receptacle in the bay. The system further includes an air duct fluidly coupled to the port of the receptacle and extending through a wall of a room in which the receptacle is arranged.

### BRIEF DESCRIPTION OF THE DRAWINGS

Other objects and advantages of the invention will become apparent upon reading the following detailed description and upon reference to the accompanying drawings in which:

FIG. 1A illustrates a side view of an example of a portable disinfection apparatus;

FIG. 1B illustrates a side view of an example of a different portable disinfection apparatus;

FIG. 2 illustrates a front perspective view of a receptacle for receiving a portable disinfection apparatus;

FIG. 3 illustrates a front perspective view of a different receptacle for receiving a portable disinfection apparatus;

FIG. 4 illustrates a front perspective view of a yet another different receptacle for receiving a portable disinfection apparatus; and

FIG. 5 illustrates a front perspective view of a yet another different receptacle for receiving a portable disinfection apparatus.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

The devices and systems described herein involve base stations having occupancy sensors and/or air disinfection capability for working in conjunction with portable area/room disinfection apparatuses. As used herein, the terms “portable” and “mobile” refer to the capability of moving or being moved and may be used interchangeably herein. Configurations to affect mobility of the disinfection apparatuses considered herein may include but are not limited to wheels (motorized or non-motorized), one or more handles, navigational program instructions (including preprogrammed paths, navigation via remote control and/or autonomous capability), a weight and design which affords the apparatus to be efficiently and safely transported at least 1 meter, or any combination thereof. It is emphasized that the disinfection apparatuses considered herein may include any one or more of such configurations in any combination to affect its mobility and, thus, the apparatuses are not limited to including all of the noted configurations. For instance, an apparatus having motorized wheels may or may not include navigational program instructions. Furthermore, an apparatus configured for autonomous movement may or may not include a handle. Moreover, an apparatus having one or more handles may or may not have wheels and vice versa. In addition, an apparatus with wheels is generally easier to push or pull than an apparatus without wheels and, thus, apparatuses considered herein having wheels may be but are not necessarily heavier than those without wheels. In any case, all references of an apparatus having wheels disclosed herein refer to an apparatus having a device of a wheel attached to an axle. In some cases, the wheels of an apparatus considered herein may be casters.

In general, the parameters constituting “a weight and design which affords the apparatus to be efficiently and safely transported at least 1 meter” may vary among apparatuses. For instance, in cases in which an apparatus is not motorized, the phrase may pertain to an ergonomic weight and design which affords the apparatus to be efficiently and safely carried, pushed and/or pulled at least 1 meter by one or more adults of average height and weight. For example, the weight of a disinfection apparatus considered herein that is not motorized may, in some cases, be less than approximately 25 pounds, particularly but not limited to if the apparatus does not include wheels and is of a design (i.e., size, shape, etc.) that one individual may manipulate the relocation of the apparatus. Alternatively, the weight of a disinfection apparatus considered herein that is not motorized may, in some cases, be more than approximately 25 pounds but less than approximately 200 pounds, particularly if the apparatus includes wheels and/or is of a design that facilitates multiple individuals to manipulate the relocation of the apparatus (e.g., includes multiple handles). In yet other embodiments, the weight of a disinfection apparatus considered herein that is not motorized and has wheels and/or has a design that multiple individuals may manipulate the relocation of may be less than approximately 25 pounds.

In alternative embodiments, the disinfection apparatuses considered herein may be motorized and, in such cases, the weight of an apparatus may not be as restricted as those which are not motorized, particularly an apparatus which is motorized may be any weight and design which affords the apparatus to efficiently and safely travel at least 1 meter. For example, a disinfection apparatus considered herein that is motorized may include a weight and motor controls which allow the movement of the apparatus to be started and stopped without undue time delay (e.g., less than 5 seconds from the receipt of signals to start and stop movement of the apparatus). In addition, a disinfection apparatus considered herein that is motorized may include a weight, speed controls, and/or navigational controls which allows the apparatus to be moved without causing damage to the apparatus or infrastructure along the path of the apparatus. For instance, a disinfection apparatus considered herein that is motorized may include navigational controls which prevent the apparatus from bombarding with obstacles and/or walls. In addition or alternatively, a disinfection apparatus considered herein that is motorized may be configured to limit the speed of the apparatus, but yet enable speeds such that an apparatus may travel to a position in a timely manner, particularly regarding the idea of the apparatuses considered herein being configured to efficiently travel at least 1 meter. An example range of speed for the disinfection apparatuses considered herein may be up to approximately 200 meter/minute, but faster speeds may be considered.

In any case, an apparatus that is motorized may include configurations for a user to guide the apparatus and/or configurations for the apparatus to autonomously guide itself. Example configurations which may enable a user to guide a mobile apparatus include one or handles and/or one or more user input controls, such as but not limited to a steering wheel, a joy stick, a means for enabling audible input for directional movement and/or one or more tactile input controls denoting particular directional movements (such as but not limited to buttons, switches, graphical user interfaces and/or touch sensor means). In some cases, configurations for enabling a user to guide an apparatus may be integrated into the apparatus such that movement of the apparatus may be controlled at the apparatus. In some of such scenarios, an apparatus may include configurations to accommodate a user guiding the apparatus, such as a seat if the mobile apparatus is a vehicle (e.g., a user driven floor cleaner) and/or shields if movement of the mobile apparatus is susceptible to exposing the user to harm. In yet other cases, user input controls for enabling a user to guide an apparatus may be integrated into a detached user interface used in conjunction with the apparatus such that movement of the apparatus may be controlled via remote control. In some cases, a mobile apparatus may include configurations integrated into the apparatus and configurations integrated into a detached user interface for enabling a user to guide its movement. In yet other cases, a mobile apparatus may be additionally or alternatively configured to guide itself, i.e., via program instructions to follow a predetermined path and/or via navigational controls for autonomous movement.

Further to the idea of the portable disinfection apparatuses described herein being configured to be efficiently transported at least 1 meter, the apparatuses considered herein (regardless of whether they are motorized or not) may, in some cases, be specific to those which do not require a significant amount of time (e.g., not more than 5 seconds) to start movement of the apparatus across an area or room. As such, the disinfection apparatuses considered herein do not embody apparatuses which are screwed or bolted to a surface for its operation. Moreover, the portable disinfection apparatuses disclosed herein may include guarding which allows the apparatus to be moved without causing damage to the apparatus or infrastructure along the path of the apparatus. For example, the portable disinfection apparatuses disclosed herein may have bumpers along an outermost periphery of the apparatus. In addition or alternatively, the portable disinfection apparatuses disclosed herein may have their fragile components arranged in protective housings and/or arranged inward from an outermost periphery of the apparatus.

As noted above, the devices and systems described herein involve base stations having occupancy sensors and/or air disinfection capability for working in conjunction with portable disinfection apparatuses. In some cases, the portable disinfection apparatuses may be specific to area/room disinfection apparatuses. More specifically, the portable disinfection apparatuses may have configurations which facilitate area/room disinfection. As used herein, the term “area/room disinfection apparatus” refers to an apparatus configured to disinfect a space which is suitable for human occupancy so as to deactivate, destroy or prevent the growth of disease-carrying microorganisms in the area. As used herein, the terms “area/room disinfection apparatus”, “area disinfection apparatus” and “room disinfection apparatus” may be used interchangeably herein. Although some of the receptacles and systems described herein may be specific for use with portable area/room disinfection apparatuses, the receptacles and systems need not be so limited. Furthermore, a portable area/room disinfection apparatus considered herein and used in the methods described herein need not include all or any of the features described below that are generally associated with area/room disinfection apparatuses.

The term “area/room”, as used herein, refers to a space in a building which is suitable for human occupancy. The phrase “a space which is suitable for human occupancy”, as used herein, refers to a space in which an adult human being of average size may comfortably occupy for at least a period of time to eat, sleep, work, lounge, partake in an activity, or complete a task therein. In some cases, spaces suitable for human occupancy may be a room, which is referred to herein as a space bounded by walls, ceiling, flooring, and one or more doors for entering and exiting the space. In other cases, a space suitable for human occupancy may be an area with less than all of the boundaries which characterize a room. Examples of spaces which are suitable for human occupancy include but are not limited to single patient rooms, multiple occupancy patient rooms, bathrooms, walk-in closets, hallways, bedrooms, offices, operating rooms, patient examination rooms, waiting and/or lounging areas, nursing stations, laboratories, clean rooms, stock/equipment rooms, work stations, cubicles, hotel rooms, meeting/party rooms, gyms, work-out rooms, locker rooms, classrooms, store merchandising floors, store aisles, library aisles and sitting areas, airplane cabins, cockpits, watercraft cabins and vehicles.

Examples of buildings which may be particularly applicable for the use of the methods and apparatuses disclosed herein include but are not limited to healthcare facilities (including but not limited to hospitals, urgent care facilities, clinics, nursing homes, outpatient surgical facilities, birth centers, dialysis centers, hospice centers and blood banks), pharmaceutical laboratories and plants, childcare facilities, fitness centers, food manufacturing and/or processing facilities, animal care centers, agricultural buildings, office buildings, stores, hotels, schools and libraries. The apparatuses and methods disclosed herein may also be used in various types of aircraft and watercraft, including but not limited to airplanes, jets, helicopters, cruise ships, boats, and submarines. In general, areas/rooms which may be considered for the systems disclosed herein as well as for use of the portable disinfection apparatuses disclosed herein may have a footprint greater than approximately 25 ft², but smaller areas/rooms may be considered.

In general, an area/room disinfection apparatus includes configurations to distribute an effective amount of germicide in a spacious manner to an ambient of an area/room in which the apparatus is arranged to maximize the number of surfaces and objects disinfected in the area/room. The apparatus may be of any shape, size, or configuration to achieve such an objective. An example configuration of an area/room disinfection apparatus which may be particularly considered for the portable disinfection apparatuses discussed herein is to be configured to direct a germicide to a region approximately 2 feet and approximately 8 feet from a floor of an area/room in which the apparatus is arranged. In particular, the region between approximately 2 feet and approximately 8 feet from a floor of a room is considered a “high touch” region of an area/room since objects of frequent use are generally placed in such a region. Examples of objects typically found in a high touch region of a room include but are not limited to desktops, keyboards, telephones, chairs, door and cabinet handles, light switches and sinks, or in other words, any object within an arm's reach. Objects in a high touch region of an area/room that are of particular interest to disinfect are objects within an arm's reach (i.e., approximately 30 inches) of a location which is designed for an individual to occupy (i.e., sit, stand, or lie down) for an extended period of time, such as but not limited to a chair/seat, a workstation (sitting or standing), a bed or an examination table.

Another feature of an area/room disinfection apparatus which may be included in the disinfection apparatuses considered herein is to include configurations to distribute an effective amount of germicide to achieve at least a 2-log reduction in bacterial contamination on surfaces that are greater than 1 meter or even 2 or 3 meters from the germicidal source. Configurations used to generate such an effect generally depend on the time of the disinfection cycle and the configuration of the germicidal source, particularly the size of the germicidal source, the intensity and/or frequency at which the germicide is dispersed and/or the orientation of the germicidal source in the apparatus. In general, the germicidal sources considered herein may be any shape, size, orientation and configuration and may be conducted at parameters to achieve a desired reduction in bacterial contamination on surfaces that are greater than 1 meter or even 2 or 3 meters from the apparatus. An example of an orientation of a germicidal source which may aid in achieving such an effect is a germicidal light source vertically arranged (e.g., the germicidal light source is arranged lengthwise substantially perpendicular to a horizontal plane of the apparatus) to aid in distributing the germicide greater distances within an area/room. Moreover, in cases in which an area/room disinfection apparatus includes a germicidal light source as its germicidal source, power fluxes of at least 1.0 W/m² may be generally used to achieve at least a 2-log reduction in bacterial contamination on surfaces within an area/room that are greater than 1 meter from the germicidal source.

As noted above, a disinfection apparatus considered herein and used in the methods described herein need not include all or any of the features that are generally associated with area/room disinfection apparatuses. As such, a disinfection apparatus considered herein may not, in some cases, emit an effective amount of germicide to achieve at least a 2-log reduction in bacterial contamination on surfaces that are greater than 1 meter from its germicidal source. In particular, it is contemplated that a disinfection apparatus having one or more of the features disclosed herein may be designed to be used in a specific environment, including but not limited to those in which most if not all objects targeted for disinfection are less than a meter away from the location the disinfection apparatus is placed for a disinfection process. As such, for such an application, a disinfection apparatus may not include a disinfection source that achieves at least a 2-log reduction in bacterial contamination on surfaces that are greater than 1 meter from the germicidal source. In some cases, germicidal sources that do not achieve at least a 2-log reduction in bacterial contamination on surfaces greater than 1 meter from the germicidal source may be cheaper and/or require less energy than those that do and, thus, it may be advantageous to employ such a germicidal source in a disinfection apparatus that is specifically designed for disinfecting objects less than 1 meter away from its germicidal source.

Another feature common to area/room disinfection apparatuses which some of the disinfection apparatuses described herein may include is one or more actuators for moving its germicidal source with respect to other components of the apparatus (such as a support structure supporting the germicidal source or a base of the apparatus) to aid in the distribution of the germicide in an area/room. In such cases, a germicidal source may be moved in vertical, horizontal and/or diagonal directions or may be rotated or oscillated via the one or more actuators. In some cases, the area/room disinfection apparatuses disclosed herein may include processor-executable program instructions for activating one or more of the actuator/s to move the germicidal source relative to other components of the apparatus while the germicidal source is emitting a germicide and/or in between projections of the germicide in cases in which a pulsed germicidal source is used. Other features of area/room disinfection apparatuses include wheels and/or a handle to affect portability for the apparatuses. In addition, many area/room disinfection apparatuses include configurations for remotely starting the apparatuses such that individuals need not be present in the area/room when operation of the apparatus commences.

Another feature of an area/room disinfection apparatus which may be included in the disinfection apparatuses considered herein is a configuration to distribute a germicide 360° around the apparatus. For example, in some cases, the arrangement of one or more germicidal sources in a disinfection apparatus may be such that a germicide emitted from the germicidal source/s is projected approximately 360° around the apparatus. In some cases, a disinfection apparatus may include a single germicidal source that is arranged to distribute a germicide approximately 360° around the apparatus. In other cases, a disinfection apparatus may include a plurality of germicidal sources that are arranged to collectively distribute a germicide approximately 360° around the apparatus. In either case, the disinfection apparatuses may be void of germicide-blocking components approximately 360° around the apparatus in the area of the germicidal sources such that the germicide emitted from the germicidal source/s substantially encircles the apparatus.

In additional or alternative cases, a disinfection apparatus may be configured to move its one or more germicidal sources and/or other components of the apparatus to distribute a germicide approximately 360° around the apparatus. For instance, in some cases, a disinfection apparatus may include a moveable germicidal source (such as but not limited to a sprayer or laser) that is programmed to move to distribute a germicide 360° around the apparatus. In other cases, a disinfection apparatus may include one or more germicidal sources in a housing that has one or more openings or one or more transparent windows to emit a germicide from the germicidal source/s to an exterior of the apparatus. In such cases, the housing and, in some cases, the germicidal sources may be moved to achieve a 360° distribution of the germicide around the apparatus through the openings and/or holes of the apparatus. In yet other cases, particularly in scenarios in which a disinfection apparatus includes a germicidal light source, the disinfection apparatus may include a reflector to help distribute germicidal light 360° around the apparatus. In such cases, the reflector may be stationary or, alternatively, may move to achieve the 360° distribution of the germicide light.

In yet other embodiments, a disinfection apparatus considered herein may not include a configuration to distribute a germicide 360° around the apparatus. In particular, for some environments it may be advantageous to focus germicide emission to a particular area of an area/room or even a particular surface. In addition or alternatively, it may be advantageous in some applications to block a germicide from reaching some areas of an environment. As such, for some disinfection apparatuses, a 360° distribution of a germicide will not be prudent. In some cases, a disinfection apparatus having one or more of the features disclosed herein may include a shield to block a germicide from reaching a particular area of an environment in which the apparatus is arranged.

Yet another feature which may be included in the disinfection apparatuses described herein is processor executable program instructions for receiving data regarding characteristics of an enclosed space in which the disinfection apparatus is to be operated. In general, the phrase “characteristics of an enclosed space” as used herein refers to physical attributes as well as non-physical attributes of an enclosed space. Non-physical attributes of an enclosed space include but are not necessarily limited to identifiers used to reference an enclosed space (e.g., room number and/or room name) and occupancy information regarding an enclosed space (e.g., infection information of a patient previously occupying the space or a patient scheduled to occupy the space). Physical attributes of an enclosed space include but are not necessarily limited to size and/or dimensions of the enclosed space and/or the number, size, distances, locations, reflectivity and/or identification of surfaces, objects and/or items within the enclosed space. In some cases, a physical attribute of an enclosed space may be the identification of one or more pathological organisms and, sometimes further the number or concentration of such organism/s in the enclosed space, in a particular region of the enclosed space, or on a particular surface in the enclosed space. In any case, the data received regarding the characteristics of the enclosed space in which the disinfection apparatus is to be operated may be utilized in a number of manners, including but not limited to recordation or reporting purposes or setting one or more operational parameters of the apparatus.

As set forth above, the portable disinfection apparatuses considered herein may, in some cases, be configured for autonomous movement. In some such cases, the apparatus may include program instructions to move the apparatus in accordance with room characteristics of a room which have been analyzed via one or more sensors of the apparatus, including sensors for mapping or modeling an area/room. In some cases, the automatic movement of a disinfection apparatus considered herein may include real-time adjustments based on feedback of its sensors. In other cases, a disinfection apparatus considered herein may be configured to detect and follow a track on the floor. In yet other cases, a disinfection apparatus may be configured to use landmarks of an area/room for its navigation, such as but not limited to arms of chairs along an aisle, arrangement of shelves in a warehouse or the emergency floor lights on an airplane or movie theatre. In yet other cases, a disinfection apparatus may be configured to record its movement while it is manually driven over a course prior to a disinfection process and then can repeat the path on its own during a disinfection process.

Regardless of their configuration or setting in which they are used, the portable disinfection apparatuses considered herein include one or more germicidal sources. The germicidal source/s may be any device configured to generate a dispersible germicide. In particular, the germicidal sources may be any device or apparatus configured to generate a germicide in the form of a liquid, a vapor, a gas, a plasma or germicidal light. As used herein, the term “germicide” refers to an agent for deactivating or killing microorganisms, particularly disease carrying and/or disease producing microorganisms (a.k.a., germs). The term “kill,” as used herein, means to cause the death of an organism. In contrast, the term “deactivate,” as used herein, means to render an organism unable to reproduce without killing. As such, a germicide which is configured to deactivate a microorganism, as used herein, refers to an agent which renders a microorganism unable to reproduce but leaves the organism alive. Furthermore, the term “germicidal source” as used herein refers to a collection of one or more components used to generate and disperse a germicide. In some embodiments, a germicidal source may include components in addition to the component/s used to generate the germicide to affect the dispersal of the germicide from the generation component/s. In any case, the device and methods described herein may utilize any number of germicidal sources, including a single germicidal source or any plurality of germicidal sources. Furthermore, in cases in which a device or method utilizes multiple germicidal sources, the multiple germicidal sources may be configured to generate the same or different germicides.

As noted above, a germicidal source utilized by a portable disinfection apparatus considered herein may be a source configured to generate germicidal light. The term “germicidal light” refers to light which is capable of deactivating or killing microorganisms, particularly disease carrying and/or disease producing microorganisms (a.k.a., germs). Ranges of light which are known to be germicidal include ultraviolet B (UVB) and ultraviolet C (UV-C) light, particularly ultraviolet light between approximately 200 nm and approximately 320 nm, and more particularly ultraviolet light at 220 nm and ultraviolet light between 260 nm and 265 nm. Another range of light which is known to be germicidal includes visible violet-blue light (also known as high-intensity narrow-spectrum (HINS) light) between approximately 400 nm and approximately 470 nm, and particularly at 405 nm. The germicidal sources considered for portable disinfection apparatuses considered herein may be configured to generate any one or more of such ranges or wavelengths of germicidal light.

In some embodiments, a germicidal light source of the portable disinfection apparatuses considered herein may generate ranges of light which are not germicidal such as but not limited to visible light greater than approximately 400 nm, but such capability will not deter from the reference of the light source being germicidal. To that regard, a light source or lamp of the portable disinfection apparatuses considered herein may, in some cases, be characterized in the type of light it generates, but such characterization need not limit the light source or lamp to generating only that type of light. For example, an ultraviolet light source is one which generates ultraviolet light but it may produce light of other wavelengths. In any case, the germicidal light source/s considered for portable disinfection apparatuses may be of any size, shape and configuration. The terms “germicidal light source” and “germicidal lamp” are used interchangeably herein and refer to a collection of one or more components used to generate and disperse germicidal light. Examples of germicidal light sources which may be configured to generate ultraviolet light and/or HINS light include discharge lamps (such as a mercury-vapor lamp or a xenon lamp), light emitting diode (LED) solid state devices, and excimer lasers. In addition, the light sources considered for the portable disinfection apparatuses considered herein may include those which generate continuous light and those which generate light in short durations, the latter of which are often referred to as flashtubes or flashlamps. Flashtubes or flashlamps that are used to supply recurrent pulses of light are often referred to as pulsed light sources.

In some cases, the portable disinfection apparatuses considered herein may utilize a germicidal source configured to generate a liquid, vapor, gaseous or plasma germicide. Examples of germicidal sources which may be configured to disperse liquid, vapor, gaseous, or plasma germicides include but are not necessarily limited to liquid sprayers, foggers, plasmas torchers and misting systems including wet and dry mist systems. An example of a gaseous germicide that is ozone. Examples of plasmas germicides are those that include reactive oxygen species. Examples of liquid and vapor germicides include solutions having a principal disinfection agent such as but not limited to bleach, hydrogen peroxide, chlorine, alcohol, quaternary ammonium compounds or ozone. In such cases, the liquid and vapor germicides may be aqueous or non-aqueous.

In addition to the many variants described above, the power source among the portable disinfection apparatuses considered for use with the receptacles disclosed herein may vary. In particular, the portable disinfection apparatuses considered for use with the receptacles disclosed herein may include a power cord and/or their own battery or, in some cases, may include a power socket for receiving a power cord. In cases in which a portable disinfection apparatus includes a battery, the apparatus may generally further include an electrical contact for mating with an electrical contact of a battery charger associated with the portable disinfection apparatus, which in some embodiments as described below may be included in the receptacles disclosed herein. The electrical contacts may take any various forms known in the art, including but not limited to electric plates and male/female connectors. In any case, the battery may generally be sufficient to power the portable disinfection apparatus to conduct multiple disinfection cycles and, preferably, over a time span of at least an hour. Due to the power requirements of conducting area/room disinfection processes, some of the portable disinfection apparatuses considered herein may include a battery having a capacity of at least 10 Ah and, in some cases, a battery having a capacity of at least 30 Ah.

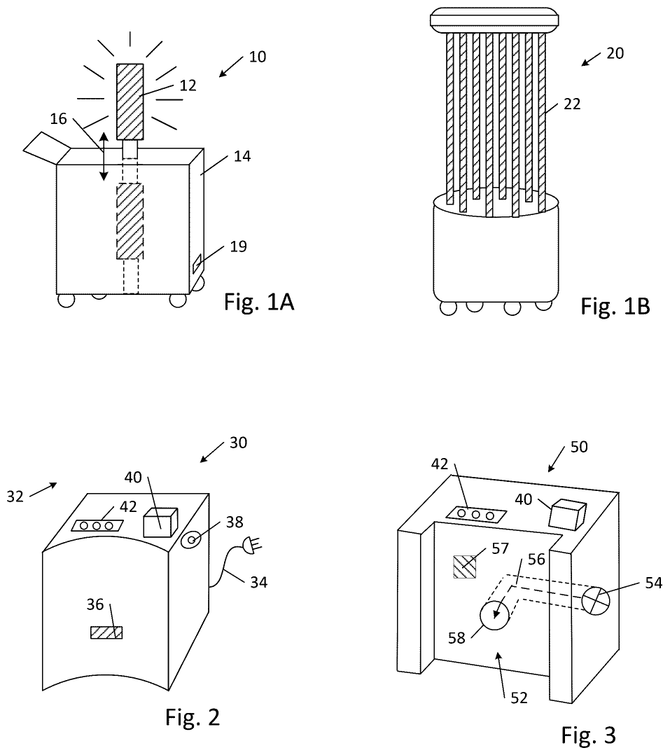

In general, the receptacles described herein may be used in conjunction with many different types of portable disinfection apparatuses. However, as set forth in more detail below in reference to FIG. 3, one of the receptacles may be specific to being used with a portable disinfection apparatus which has a germicidal source arranged within an interior chamber of the apparatus. In some cases, the germicidal source may be fixedly arranged in the interior chamber of the portable disinfection apparatus. In other cases, the portable disinfection apparatus may be configured to move a germicidal source in and out of the chamber such that the germicidal source may be used to conduct a disinfection process exterior to the apparatus and interior to the apparatus. An example of a portable disinfection apparatus having such a configuration is shown in FIG. 1A and may be considered for use with any the receptacles disclosed herein. In particular, FIG. 1A illustrates portable disinfection apparatus 10 having germicidal source 12 disposed above chamber 14. As shown by the doubled arrow line 16 shown in FIG. 1B, germicidal source 12 may be supported and moveable in and out of chamber 14 via support members 18. In particular, support members 18 may be coupled to a lower portion of germicidal source 12 and may be configured to draw germicidal source 12 into chamber 14 as denoted by the dotted line version of germicidal source 12 in FIG. 1A. In some cases, an actuator (i.e., a motorized component) may be used to affect automated movement of germicidal source 12. However, in other cases, movement of germicidal source 12 may be manually affected by a user of apparatus 20. In any case, although portable disinfection apparatus 10 is shown having a single germicidal source, it may alternatively include multiple germicidal sources.

Alternative to the portable disinfection apparatus shown in FIG. 1A, a different type of portable disinfection apparatus which may be considered for use in conjunction with most of the receptacles disclosed herein has one or more germicidal source fixedly arranged to project a germicide exterior to the apparatus. An example of a portable disinfection apparatus having such a configuration is shown in FIG. 1B. In particular, FIG. 1B illustrates portable disinfection apparatus 20 having germicidal light sources 22 vertically and fixedly disposed between a wheeled base and a top support. As shown, the germicidal light sources are arranged such that germicidal light may be projected into an ambient of the apparatus.

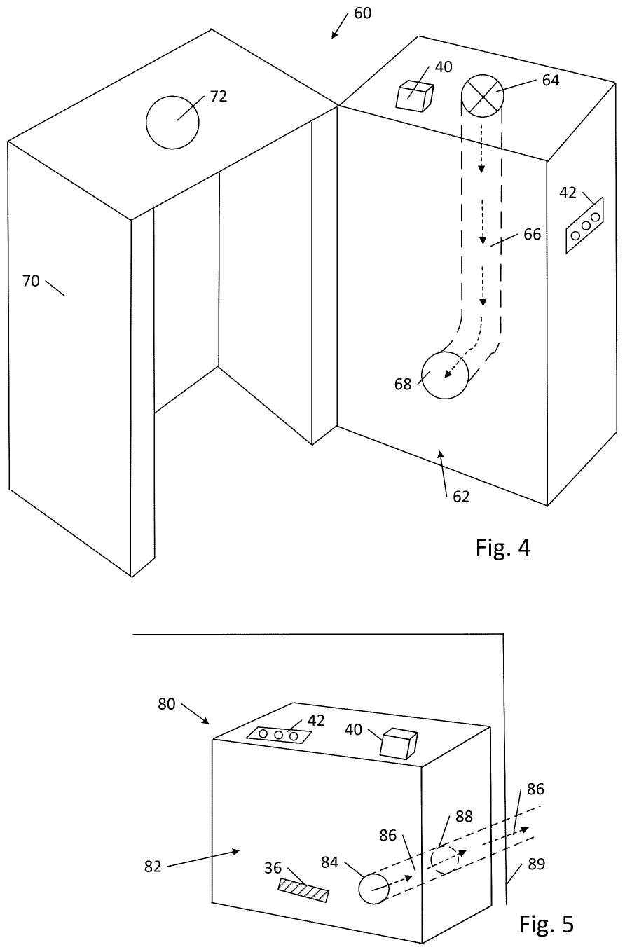

As noted above, the receptacles described herein may be used in conjunction with many different types of portable disinfection apparatuses and, thus, the receptacles described herein may be used with the disinfection apparatuses having different configurations than shown and described in reference to FIGS. 1A and 1B. In particular, the portable disinfection apparatuses considered applicable for the receptacles described herein are not limited to apparatuses having a germicidal source extending beyond a support structure of the apparatus as shown and described in reference to FIGS. 1A and 1B. Rather, a portable disinfection apparatus considered applicable for the receptacles described herein may, in some embodiments, have a germicidal source arranged partially or wholly inset to a structure of the apparatus with an opening or transparent window to allow the transmission of a germicide generated therefrom to be projected into an ambient of the mobile apparatus. In addition or alternatively, a portable disinfection apparatus considered applicable for the receptacles described herein may have a germicidal source arranged along any portion of the apparatus, including the top portion, sidewall portions or the bottom of the apparatus. Many other configurations of portable disinfection apparatuses may be considered. In any case, with exception of the receptacle disclosed in reference to FIG. 4, the receptacles disclosed herein may generally be smaller than the portable disinfection apparatuses they are configured to receive.

Turning to FIG. 2, an example configuration of a receptacle for storing a portable disinfection device is shown. In particular, FIG. 2 illustrates receptacle 30 having bay 32 for receiving a portable disinfection apparatus. In general, bay 32 may be any shape, size, or configuration to accommodate a particular portable disinfection device and, thus, should not be construed to be limited to the depiction in FIG. 2. For example, bay 32 may alternatively include a configuration of one of the bays shown for the receptacles depicted FIGS. 3-5. Other bay configurations may also be considered. As such, the configuration of bay 32 is not mutually exclusive to the features described in reference to receptacle 30. Furthermore, the size, shape, and configuration of receptacle 30 as a whole may vary and, thus, is not restricted to the depiction in FIG. 2. As shown, receptacle 30 may include power cord 34, or alternatively its own battery or a power socket for receiving a power cord, such that power may be supplied to electrical components of the device, such as but not limited to sensors 38, controller 40, and user interface 42, which are explained in more detail below.

In cases in which the portable disinfection device used in conjunction with receptacle 30 includes a battery, receptacle 30 may, in some embodiments, include electrical contact 36 arranged along an exterior surface of bay 32 for mating with a power charging contact of a portable disinfection apparatus. In this manner, receptacle 30 may serve as a charging station for the portable disinfection apparatus. In general, electrical contact 36 may be of any form known in the art, including but not limited to electric plates and male/female connectors. In further of such cases, receptacle 30 may include a battery charger device configured to regulate the amount of current discharged from electrical contact 36. Due to the power requirements of portable area/room disinfection apparatuses (and thus the size of their battery), a battery charger of receptacle 30 may generally be configured to discharge at least 4.0 amps through electrical contact 36 in order to provide a charging time of a few hours. In yet other cases, however, receptacle 30 may not be a charging station for the portable disinfection apparatus it is configured to receive and, thus, receptacle 30 may not include a battery charger or electrical contact 36 (i.e., receptacle 30 may be void of electrical charging contacts along the receiving surfaces of bay 32). Furthermore, the portable disinfection apparatus which bay 32 is configured to receive need not include a battery, but rather may include a power cord or a power socket for receiving a power cord.

As further shown in FIG. 2, receptacle 30 includes sensors 38. Sensors 38 are configured to detect a parameter in the room which is indicative of occupancy or potential occupancy of a human or an animal in a room or area. The parameter may be motion, heat, radar, or an image. Detecting any of such parameters within an interior of a room may be indicative of occupancy in the room and, as such, a sensor arranged and used to monitor an interior space of a room other than door movement is referred to herein as an occupancy sensor. Detecting movement of a door to a room may be indicative of potential occupancy in the room and, thus, a sensor arranged used to monitor movement of a door is referred to herein as a door movement sensor. Examples of occupancy sensors which may be included on receptacle 30 (as well as any of the other receptacles disclosed herein) include but are not limited to motion sensors, thermal sensors, Doppler sensors, or photo recognition sensors. Examples of door movement sensors which may be included on receptacle 30 (as well as any of the other receptacles disclosed herein) include but are not limited to motion sensors and proximity sensors. Although receptacle 30 is shown including two sensors 38, any number of sensors may be used, including a single sensor or any multiple of sensors. In the latter embodiment, the multiple sensors may be of the same type or may be of different types.

Furthermore, sensors 38 may be arranged at different locations of receptacle 30 than shown in FIG. 2, such as but not limited to the upper surface of the receptacle or along lower portions of its sidewalls. Although it may be advantageous to have sensors 38 arranged on different sides of receptacles to be able to monitor areas on either side of the receptacle, in some cases, sensors 38 may be arranged along the same surface of receptacle. In some embodiments, one or more of sensors 38 may be repositionable. For instance, in some cases, one or more sensors 38 may be pivotally secured to the receptacle such that the angle at which the sensor is directed may be manipulated. In addition or alternatively, one or more sensors 38 may be detachable from receptacle 30 and receptacle 30 may include other locations at which to readily re-attachable the sensor/s. In this manner, the one or more sensors may be directed at one or more particular areas to monitor as selected by a user of the receptacle, which may differ depending on the layout of the room in which the receptacle is used. For example, in an embodiment in which receptacle 30 includes a repositionable door movement sensor, the door movement sensor may be arranged such that it is directed toward a door in the room. Alternatively, in some cases, receptacle 30 may be arranged such that a door movement sensor thereon is in alignment with a door of the room in order to detect movement of the door. In some of such cases, receptacle 30 may be positioned within approximately one foot of a door of a room to ensure a high degree of door movement detection.

In order for sensors 38 to affect the operation of a portable disinfection apparatus in the room, receptacle 30 includes a wireless transmitter communicably coupled to sensors 38 for sending a signal into an ambient of the receptacle upon sensors 38 detecting occupancy or door movement in the area/room. In particular, to prevent inadvertent exposure of a germicide to individuals during disinfection of an area/room, the wireless transmitter of receptacle 30 sends a signal into an ambient of the area/room upon detecting door movement and/or occupancy in the area/room. The signal is specific to the portable disinfection apparatus which receptacle 30 is configured to accommodate and serves to command the portable disinfection apparatus to inhibit or terminate activation of a power supply circuit to its germicidal source/s. In order to affect such communication, the portable disinfection apparatus includes a wireless transceiver. In some cases, the wireless transmitter of receptacle 30 is configured to send a different signal into an ambient of the room to activate the germicidal source/s of the portable disinfection apparatus after a predetermined duration of sensors 38 not detecting door movement and/or occupancy in the area/room. Such a signal may be particularly applicable when a disinfection process is initiated for the area/room and, as a safety precaution, a certain amount of time is lapsed to ensure there is no human or animal presence in the room prior to activating the germicidal source/s of the portable disinfection apparatus. In any case, an advantage of receptacle 30 including sensors 38 and a wireless transmitter communicably coupled to the sensors is the portable disinfection apparatus used the presence of receptacle 30 need not include an occupancy sensor and/or a door movement sensor.

The wireless transmitter coupled to sensors 38 may be incorporated in controller 40 or may be independently arranged on receptacle 30. In general, controller 40 is configured to affect operations of receptacle 30, including but not limited to sending instruction for the wireless transmitter to send signal/s, processing signals from user interface 42, controlling activation of sensors 38 and, if applicable, supplying power to electrical contact 36 upon detecting contact thereto. In some embodiments, user interface 42 may include an input terminal to activate sensors 38. In other embodiments, receptacle 30 may include a sensor for detecting if bay 32 is occupied (i.e., a sensor different from sensors 38). The sensor for detecting if bay 32 is occupied may be incorporated into electrical contact 36 or may be arranged on a surface of receptacle 30 in or in proximity to bay 32. Examples of a sensor for detecting if bay 32 is occupied include but is not limited a touch sensor or proximity sensor.

In any case, controller 40 may include program instructions for inhibiting activation of sensors 38 when the additional sensor detects bay 32 is occupied and also activating sensors 38 subsequent to the additional sensor detecting the bay is not occupied. In this manner, the sensors will not be activated when the portable disinfection apparatus is received within receptacle 30 since the portable disinfection apparatus in such a position is not likely to be used for a room/area surface disinfection process. In yet other cases, sensors 38 may be continually activated. In yet other embodiments, the portable disinfection apparatus which receptacle 30 is configured to receive may include a sensor for detecting if the apparatus is seated in bay 32. The sensor may be incorporated into an electrical charging contact on the portable disinfection apparatus or may be arranged on a surface of portable disinfection apparatus which is to be received into bay 32. In either of such cases, in order to affect the communication of the sensor signals, the portable disinfection apparatus includes a wireless transmitter and receptacle 30 includes a wireless receiver.

In any case, the use of receptacle 30 with a particular portable disinfection apparatus constitutes a system. The system may, in some embodiments, be configured to transport the receptacle and portable disinfection apparatus together such that they may be used in various areas and rooms. In some cases, the portable disinfection apparatus may be one of a plurality of portable disinfection apparatus which may be received within bay 32 of receptacle 30 and which is configured to receive signals from the wireless transmitter of receptacle 30. In addition of alternatively, receptacle 30 may be one of a plurality of receptacles which may be configured to receive and communicate with the portable disinfection apparatus. In this manner, the plurality of receptacles may each be placed in a separate room or area, enabling the portable disinfection apparatus to safely perform disinfection processes within each of the rooms/areas.

Turning to FIG. 3, a different configuration of a receptacle for storing a portable disinfection device is shown and, particularly one which is configured to provide air disinfection in a room while the portable disinfection device is arranged at the receptacle. In particular, FIG. 3 illustrates receptacle 50 having bay 52 for receiving a portable disinfection apparatus. In general, bay 52 may be any shape, size, or configuration to accommodate a particular portable disinfection device and, thus, should not be construed to be limited to the depiction in FIG. 3. As such, the configuration of bay 52 is not mutually exclusive to the features described in reference to receptacle 50. Bay 52 is shown having a different configuration than bay 32 of receptacle 30 shown in FIG. 2 to illustrate that the configuration of bays may differ, depending on the design specifications of the portable disinfection apparatus to be received therein. Furthermore, the size, shape, and configuration of receptacle 50 as a whole may vary and, thus, is not restricted to the depiction in FIG. 3.

Similar to receptacle 30 shown in FIG. 2, receptacle 50 may include a power cord, or alternatively its own battery or a power socket for receiving a power cord, such that power may be supplied to electrical components of the device, such as but not limited to controller 40, user interface 42 and, if applicable, sensors 38. Such components are not shown in receptacle 50 of FIG. 3 to simplify the drawing, but receptacle 50 may include one or more of such features nonetheless. The descriptions of controller 40, user interface 42 and sensors 38 in reference to receptacle 30 of FIG. 2 are equally applicable to receptacle 50 depicted in FIG. 3. In cases in which the portable disinfection device to be received by bay 52 includes a battery, receptacle 50 may, in some embodiments, include electrical contact 36 arranged along an exterior surface of bay 52 for mating with a power charging contact of a portable disinfection apparatus. In this manner, receptacle 50 may serve as a charging station for the portable disinfection apparatus. In some of such cases, receptacle 50 may include a battery charger device configured to regulate the amount of current discharged from electrical contact 36. In yet other cases, receptacle 50 may not be a charging station for the portable disinfection apparatus it is configured to receive and, thus, receptacle 50 may not include a battery charger or electrical contact 36. Furthermore, the portable disinfection apparatus which bay 52 is configured to receive need not include a battery.

As further shown in FIG. 3, receptacle 50 includes air moving device 54 disposed within an air inlet along the side of receptacle 50 for drawing air from an ambient of the receptacle. In addition, receptacle 50 is shown with air channel 56 coupled between the air inlet and air outlet 58 along a surface of receptacle 50 in bay 52. To such a regard, the portable disinfection apparatus to be received in bay 52 includes an air inlet along its exterior surface dimensionally configured and arranged to be connected to air outlet 58 of receptacle 50. Portable disinfection apparatus 20 depicted in FIG. 1B includes air inlet 29 along the side of its carriage as an example of an apparatus configuration which may mate with receptacle 50 to provide an air channel from air moving device 54 to air inlet 29 of portable disinfection apparatus 20 when the portable disinfection apparatus is arranged in bay 52. Although air moving device 54 is shown along the right-side external panel of receptacle 50, the position of the air moving device is not so limited. In particular, air moving device 54 may be arranged in any location along air channel 56. Furthermore, the location and configuration of air channel 56 as well as air outlet 58 and the air inlet to the channel are not limited to the depiction in FIG. 3. For instance, air outlet 58 and the air inlet to air channel 56 need not be at the same elevational height in receptacle 50. Furthermore, the air inlet to air channel 56 may alternatively be on a different side of receptacle 50 or along the upper surface of receptacle 50. Furthermore, air outlet 58 may be at any location of the surface of receptacle 50 in bay 52. Regardless of the configuration of such features, receptacle 50 may include an air filter along air channel 56.

In any case, routing ambient air through receptacle 50 to a portable disinfection apparatus received in bay 52 offers a manner to use the portable disinfection apparatus as an air disinfection device without requiring the portable disinfection apparatus to include an air moving device or an air filter. Furthermore, in cases in which the portable disinfection apparatus is being charged in bay 52 via electrical contact 36, the disinfection apparatus may be charged while performing an air disinfection process. In addition to having an air inlet to connect with air outlet 58 of receptacle, the portable disinfection apparatus includes an internal area to which its germicidal source is configured to emit a germicide. As described above in reference to FIG. 1B, portable disinfection apparatus 20 is configured to move germicidal source 22 in and out of chamber 24 via support members 28. As a consequence, the interior of chamber 24 offers an internal area of the portable disinfection apparatus to which germicidal source 22 emits a germicide and, thus, air routed to such an area can be disinfected.

It is noted that other configurations of portable disinfection apparatuses that have an air inlet and that are configured to generate a germicide within its interior may be used with receptacle 50 and, thus, the use of receptable 50 is not limited to the portable disinfection apparatus illustrated in FIG. 1B. In some cases, a portable disinfection apparatus having LEDs as a germicidal source, and particularly having LEDs arranged around a cylindrical core, may be particularly suitable for configurations of receptacle 50 since a cooling system for those LEDs would draw air internal to the core and not exterior to the LEDs. As such, a portable disinfection apparatus having such a configuration of LEDs may not have a configuration by which to perform air disinfection on its own and, thus, corroborating with receptacle 50 to produce an air disinfection system may be attainable.

In addition to the aforementioned features, the portable disinfection apparatus received in bay 52 includes an air outlet for discharging the air disinfected therein. An example of a location for an air outlet for portable disinfection apparatus 20 shown and described in reference to FIG. 1B may be the opening within chamber 24 which germicidal source 22 passes through when moving in and out of the chamber. Chamber 24, however, may include an air outlet along a different portion of the chamber. In any case, with an air outlet, the portable disinfection apparatus received in bay 52 includes an air passage between the air inlet and air outlet that comprises the internal area to which its germicidal source emits a germicide and, thus, the portable disinfection apparatus includes an air passage by which to disinfect air.

Turning to FIG. 4, a different configuration of a receptacle is shown for providing air disinfection in a room while the portable disinfection device is arranged at the receptacle. In particular, FIG. 4 illustrates receptacle 60 having bay 62 for receiving a portable disinfection apparatus. In general, bay 62 may be any shape, size, or configuration to accommodate a particular portable disinfection device and, thus, should not be construed to be limited to the depiction in FIG. 4. Bay 62 is shown having a different configuration than bays 32 and 52 of receptacles 30 and 50 shown in FIGS. 2 and 3 to illustrate that the configuration of bays may differ, depending on the design specifications of the portable disinfection apparatus to be received therein. In particular, bay 62 is shown without any indentation, but rather a flat panel which a portable disinfection apparatus may be brought in contact with or in proximity to. As described for receptacles 30 and 50 in reference to FIGS. 2 and 3 above, the size, shape, and configuration of receptacle 60 as a whole may vary and, thus, is not restricted to the depiction in FIG. 4.

Similar to receptacle 30 shown in FIG. 2, receptacle 60 may include a power cord, or alternatively its own battery or a power socket for receiving a power cord, such that power may be supplied to electrical components of the device, such as but not limited to controller 40, user interface 42 and, if applicable, sensors 38. Such components are not shown in receptacle 60 of FIG. 4 to simplify the drawing, but receptacle 60 may include one or more of such features nonetheless. The descriptions of controller 40, user interface 42 and sensors 38 in reference to receptacle 30 of FIG. 2 are equally applicable to receptacle 60 depicted in FIG. 4. In cases in which the portable disinfection device to be received by bay 62 includes a battery, receptacle 60 may, in some embodiments, include electrical contact 36 arranged along an exterior surface of bay 62 for mating with a power charging contact of a portable disinfection apparatus. In this manner, receptacle 60 may serve as a charging station for the portable disinfection apparatus. In some of such cases, receptacle 60 may include a battery charger device configured to regulate the amount of current discharged from electrical contact 36. In yet other cases, receptacle 60 may not be a charging station for the portable disinfection apparatus it is configured to receive and, thus, receptacle 60 may not include a battery charger or electrical contact 36. Furthermore, the portable disinfection apparatus which bay 62 is configured to receive need not include a battery.

As shown in FIG. 4, receptacle 60 includes air moving device 64 disposed within an air inlet along the upper surface of receptacle 60 for drawing air from an ambient of the receptacle. In addition, receptacle 60 is shown with air channel 66 coupled between air moving device 64 and air outlet 68 along a surface of receptacle 60 in bay 62. Although air moving device 64 is shown along the upper surface of receptacle 60, the position of the air moving device is not so limited. In particular, air moving device 64 may be arranged in any location along air channel 66. Furthermore, the location and configuration of air channel 66 as well as air outlet 68 and the air inlet to the channel are not limited to the depiction in FIG. 4. For instance, air outlet 68 and the air inlet to air channel 66 need not be in vertical alignment in receptacle 60. Furthermore, the air inlet to air channel 56 may alternatively be on a sidewall of receptacle 60. Furthermore, air outlet 58 may be at any location of the surface of receptacle 50 in bay 52. Regardless of the configuration of such features, receptacle 50 may include an air filter along air channel 56.

As further shown in FIG. 4, receptacle 60 includes movable encasement 70 for enclosing a portable disinfection apparatus in bay 62. More specifically, movable encasement 70 is dimensionally configured to surround a portable disinfection apparatus received at bay 62. In this manner, receptacle is configured to provide an air space around the portable disinfection apparatus received at bay 62. To such a regard, the portable disinfection apparatus to be received in bay 62 is configured to emit a germicide external to the apparatus and, thus, may treat air introduced into the space by air outlet 68. Portable disinfection apparatus 10 depicted in FIG. 1A is an example of an apparatus which may be suitable for such a receptacle configuration. In particular, as noted above, portable disinfection apparatus 10 is configured to emit a germicide into an ambient of the apparatus. It is noted that other configurations of portable disinfection apparatuses that are configured to emit a germicide into its ambient may be used with receptacle 60 and, thus, the use of receptable 60 is not limited to the portable disinfection apparatus illustrated in FIG. 1A. For instance, receptacle 60 may be used with portable disinfection apparatus 20 described in reference to FIG. 1B when germicidal source 22 is retained outside chamber 22 when brought into bay 62.

In any case, instead of routing air through a portable disinfection apparatus to disinfect air as described in reference to receptacle 50 in FIG. 3, the configuration of receptacle 60 with enclosure 70 and air channel 66 offers a manner to disinfect air within the receptacle in the ambient of the portable disinfection apparatus received therein. With such a configuration, enclosure 70 includes air outlet 72 for discharging the treated air. Although air outlet 72 is shown along the upper surface of enclosure 70, the position of the air outlet is not so limited. In particular, air outlet 72 may be arranged in any location of enclosure 72 or the adjoining portion of receptacle 60 which houses air channel 66. To such a regard, air moving device 64, air channel 66, air outlet 68 as well as the air inlet to air channel 66 may alternatively be arranged in enclosure 72.

Turning to FIG. 5, a different configuration of a receptacle for storing a portable disinfection device is shown. In particular, FIG. 5 illustrates receptacle 80 having bay 82 for receiving a portable disinfection apparatus. In general, bay 82 may be any shape, size, or configuration to accommodate a particular portable disinfection device and, thus, should not be construed to be limited to the depiction in FIG. 5. Bay 82 is shown having a different configuration than bays 32 and 52 of receptacles 30 and 50 shown in FIGS. 2 and 3 to illustrate that the configuration of bays may differ, depending on the design specifications of the portable disinfection apparatus to be received therein. In particular, bay 82 is shown without any indentation, but rather a flat panel which a portable disinfection apparatus may be brought in contact with or in proximity to. As described for receptacles 30 and 50 in reference to FIGS. 2 and 3 above, the size, shape, and configuration of receptacle 80 as a whole may vary and, thus, is not restricted to the depiction in FIG. 5.

Similar to receptacle 30 shown in FIG. 2, receptacle 80 may include a power cord, or alternatively its own battery or a power socket for receiving a power cord, such that power may be supplied to electrical components of the device, such as but not limited to sensors 38, controller 40, and user interface 42. Such components are not shown in receptacle 80 of FIG. 5 to simplify the drawing, but receptacle 80 may include one or more of such features nonetheless. In cases in which the portable disinfection device to be received by bay 82 includes a battery, receptacle 80 may, in some embodiments, include electrical contact 36 arranged along an exterior surface of bay 82 as shown in FIG. 5 for mating with a power charging contact of a portable disinfection apparatus. In this manner, receptacle 80 may serve as a charging station for the portable disinfection apparatus. In some of such cases, receptacle 80 may include a battery charger device configured to regulate the amount of current discharged from electrical contact 36. In yet other cases, receptacle 80 may not be a charging station for the portable disinfection apparatus it is configured to receive and, thus, receptacle 80 may not include a battery charger or electrical contact 36. Furthermore, the portable disinfection apparatus which bay 82 is configured to receive need not include a battery.

As shown in FIG. 5, receptacle 80 includes port 84 along a surface of the receptacle in bay 82, port 86 along a backside of receptacle 62, and air duct 86 fluidly coupled to port 84 and extending through port 88 and wall 89 of a room in which the receptacle is arranged. In some cases, receptacle 62 may be abutted against wall 89. In some of such cases, receptacle 80 may not include a backside and, thus, may not include port 88. In general, air duct 86 provides a manner to discharge disinfected air outside of the room in which receptacle 80 is arranged when a portable disinfection apparatus is received in bay 82, which as a whole provides a negative pressure air purifying unit. In some cases, receptacle 80 may include an air inlet along its exterior surface, an air moving device and an air outlet along the surface of receptacle 80 to provide an air channel by which to introduce ambient air into the receptacle for disinfection by a portable disinfection apparatus received in bay 82. Such components as well as receptacle 80 may include any of the configurations described above in reference to receptacles 50 and 60 for routing and disinfecting air via such units.

A variation to the configurations described for receptacle 50, however, is that the outlet of the portable disinfection apparatus received in bay 82 would need to be arranged to be connected to port 84 such that the air disinfected by the portable disinfection may be routed through air duct 86. Furthermore, a variation to the configurations described for receptacle 60 is that outlet 72 could be omitted since the air disinfected by the portable disinfection within enclosure would be routed through port 84 and air duct 86. In yet other cases, the portable disinfection apparatus received in bay 82 may be configured to conduct air disinfection on its own, specifically including its own air inlet and air moving device for routing ambient air into a chamber of the apparatus at which its germicidal source can emit a germicide to disinfect the incoming air. In such cases, receptacle 80 may not include an air inlet and air moving device for introducing ambient air into the portable disinfection apparatus. The portable disinfection apparatus, however, will include an air outlet arranged to be coupled to port 84 such that treated air may be discharged through air duct 86.

In some cases, a system of a portable disinfection apparatus received into receptacle 80 may be configured to additionally provide a positive pressure air purifying unit and further offer selectivity to whether to utilize the positive pressure option or the negative pressure option. In particular, the portable disinfection apparatus received in bay 82 may have an air outlet for discharging air into the room in which receptacle 80 is arranged such as described in reference to receptacle 50 of FIG. 3. In addition or alternatively, receptacle 80 may include an outlet to discharge air into the room in which it is arranged, such as described for outlet 72 of receptacle 60 in FIG. 4. In either case, receptacle 80 may optionally include an air channel by which to introduce ambient air to be disinfected by the portable disinfection apparatus which is received in its bay, such as described for air channels 56 and 66 in FIGS. 3 and 4. In any case, as noted, receptacle 80 may offer selectivity to whether to utilize its positive pressure air purifying feature or its negative pressure air purifying feature. One manner to achieve such selectively is for receptacle 80 to include a valve and associated program instructions for selectively closing port 84, port 88, or air duct 86. Alternatively, receptacle 80 may include two air channels for discharging disinfected air, one which is coupled to air duct 86 and another which is coupled to an air outlet of receptacle 80 that discharged disinfected air into the room in which the receptacle is arranged. In such cases, receptacle 80 may include one or more valves for selectively closing the channels. Regardless of whether receptacle 80 includes configurations for a positive pressure air purifying unit, receptacle may include an air moving device in port 84, port 88 or air duct 86 for drawing air from the port through the air duct.

It will be appreciated to those skilled in the art having the benefit of this disclosure that this invention is believed to provide receptacles and systems utilizing occupancy sensors and/or air disinfection capability in conjunction with portable disinfection apparatuses. Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims. The term “approximately” as used herein refers to variations of up to +/−5% of the stated number.

Various embodiments of the receptacles, systems and methods disclosed herein are discussed below as embodiment E1 to embodiment E25.
E1. A receptacle statically arranged within one foot of a door of a room, wherein the receptacle comprises:
   a bay for receiving a portable disinfection apparatus;
   a door movement sensor, wherein the receptacle is arranged in the room such that the door movement sensor is facing the door of the room; and
   a wireless transmitter communicably coupled to the door movement sensor for sending a signal into an ambient of the room upon the door movement sensor detecting occupancy or door movement.
E2. A receptacle for storing a portable disinfection apparatus, comprising:
   a bay for receiving the portable disinfection apparatus;
   a sensor, wherein the sensor is an occupancy sensor or a door movement sensor; and
   a wireless transmitter communicably coupled to the sensor for sending:
      a sensing signal into an ambient of the room upon the sensor detecting occupancy or door movement; and
      a non-sensing signal into an ambient of the room after a predetermined duration of the sensor not detecting occupancy or door movement.
E3. A receptacle for storing a portable disinfection apparatus, comprising:
   a bay for receiving the portable disinfection apparatus;
   a first sensor, wherein the first sensor is an occupancy sensor or a door movement sensor;
   a wireless transmitter communicably coupled to the first sensor for sending a signal into an ambient of the room upon the first sensor detecting occupancy or door movement;
   a second sensor for detecting if the bay is occupied by the portable disinfection apparatus; and
   a controller for:
      inhibiting activation of the first sensor when the second sensor detects the bay is occupied; and
      activating the first sensor subsequent to the second sensor detecting the bay is not occupied.
E4. A receptacle for storing a portable disinfection apparatus, comprising:
   a bay for receiving the portable disinfection apparatus;
   a sensor, wherein the sensor is an occupancy sensor or a door movement sensor;
   a wireless transmitter communicably coupled to the sensor for sending a signal into an ambient of the room upon the sensor detecting occupancy or door movement;
   and a power cord, a power socket, or a battery;
   one or more electrical contacts arranged along an exterior surface of the bay for mating with a power charging contact of the portable disinfection apparatus; and
   a battery charger device configured to discharge at least 4.0 amps through the one or more electrical contacts.
E5. A receptacle for storing a portable disinfection apparatus, comprising:
   a bay for receiving the portable disinfection apparatus, wherein the bay is void of electrical contacts for charging the portable disinfection apparatus;
   a sensor, wherein the sensor is an occupancy sensor or a door movement sensor; and
   a wireless transmitter communicably coupled to the sensor for sending a signal into an ambient of the room upon the sensor detecting occupancy or door movement.
E6. The receptacle of any of E1-E5, further comprising a user interface having an input terminal to activate the sensor.
E7. The receptacle of any of E1-E3 or E5, further comprising:
   a power cord, a power socket, or a battery; and
   an electrical contact arranged along an exterior surface of the bay for mating with a power charging contact of the portable disinfection apparatus.
E8. The receptacle of any of E1-E4, wherein the bay is void of electrical contacts for charging the portable disinfection apparatus.
E9. A system, comprising:
   a portable disinfection apparatus; and
   a receptacle for the portable disinfection apparatus, wherein the receptacle comprises any of the receptacle of E1-E6.
E10. The system of E9, wherein the portable disinfection apparatus comprises a battery having a capacity of at least 10 ampere hours.
E11. A system, comprising:
   a portable disinfection apparatus comprising a battery having a capacity of at least 10 ampere hours; and
   a receptacle for the portable disinfection apparatus, wherein the receptacle comprises:
      a first sensor, wherein the first sensor is an occupancy sensor or a door movement sensor; and
      a wireless transmitter communicably coupled to the first sensor for sending a signal to inhibit or terminate operation of the portable disinfection apparatus upon the first sensor detecting occupancy or door movement.
E12. The system of E9-E11, wherein the receptacle is smaller than the portable disinfection device.
E13. A system, comprising:
   a portable disinfection apparatus comprising:
      a germicidal source;
      an air inlet along its exterior surface;
      an air outlet along its exterior surface; and
      an air passage between the air inlet and air outlet that comprises an area internal to the portable disinfection apparatus to which the germicidal source is configured to emit a germicide; and
   a receptacle for the portable disinfection apparatus, wherein the receptacle comprises:
      an air moving device for drawing air from an ambient of the receptacle; and
      a bay for receiving the portable disinfection apparatus, wherein the receptacle is configured to provide an air channel from the air moving device to the air inlet of the portable disinfection apparatus when the portable disinfection apparatus is arranged in the bay.
E14. The system of E13, wherein the portable disinfection apparatus further comprises an actuator for moving the germicidal source to a position to emit a germicide to an area external to the portable disinfection apparatus.
E15. A system, comprising:
   a portable disinfection apparatus comprising a germicidal source arranged to emit a germicide to an area external to the portable disinfection apparatus; and
   a receptacle for the portable disinfection apparatus, wherein the receptacle comprises:
      an air moving device for drawing air from an ambient of the receptacle; and
      a bay for receiving the portable disinfection apparatus, wherein the receptacle is configured to enclose the portable disinfection apparatus in the bay and provide an air channel from the air moving device to an ambient of the portable disinfection apparatus when the portable disinfection apparatus is arranged in the bay.
E16. A system, comprising:
   a receptacle for housing a portable disinfection apparatus, wherein the receptacle comprises:
      a bay for receiving the portable disinfection apparatus; and
      a port along a surface of the receptacle in the bay; and
      an air duct fluidly coupled to the port of the receptacle and extending through a wall of a room in which the receptacle is arranged.
E17. The system of E16, wherein the air duct comprises an air moving device for drawing air from the port through the air duct.
E18. The system of E16 or E17, wherein the receptacle comprises an air moving device for drawing air from an ambient of the receptacle.
E19. The system of any of E16-E18, further comprising a valve for selectively closing the port or the air duct.
E20. The system of any of E16-E18, wherein the receptacle comprises:
   an air outlet for discharging air into the room;
   a first air channel coupled between the port and the air outlet;
   a second air channel coupled between the first air channel and the air duct; and
   one or more valves for selectively closing the first air channel or the second air channel.
E21. The system of any of E13-E20, wherein the receptacle further comprises:
   a power cord, a power socket, or a battery; and
   an electrical contact for mating with a power charging contact of the portable disinfection apparatus.
E22. The system of any of E13-E21, wherein the receptacle further comprises:
   a sensor comprising a human or animal occupancy sensor or a door movement sensor; and
   a wireless transmitter communicably coupled to the first sensor for sending a signal to inhibit or terminate operation of the portable disinfection apparatus upon the sensor detecting occupancy or door movement.
E23. The system of any of E7-E22, wherein the portable disinfection apparatus is configured for autonomous movement in a room or area in which the system is arranged.
E24. The system of any of E7-E23, wherein the germicidal source is a germicidal light source and is configurable to emit the germicidal light external to the portable disinfection apparatus.
E25. The system of any of E7-E24, wherein the germicidal source comprises a plurality of a germicidal light emitting diodes.

What is claimed is:

1. A receptacle for storing a portable disinfection apparatus, comprising:
a bay for receiving the portable disinfection apparatus;
a sensor, wherein the sensor is an occupancy sensor or a door movement sensor;
a wireless transmitter communicably coupled to the sensor for sending a signal into an ambient of a room in which the receptacle is arranged upon the sensor detecting occupancy or door movement;
a power cord, a power socket, or a battery;
one or more electrical contacts arranged along an exterior surface of the bay for mating with a power charging contact of the portable disinfection apparatus; and
a battery charger device configured to discharge at least 4.0 amps through the one or more electrical contacts.

2. The receptacle of claim 1, further comprising a user interface having an input terminal to activate the sensor.

3. The receptacle of claim 1, wherein the sensor is pivotally secured to the receptacle.

4. The receptacle of claim 1, wherein the sensor is detachable from the receptacle, and wherein the receptacle comprises multiple locations at which to readily attach the sensor.

5. The receptacle of claim 1, wherein the wireless transmitter is further for sending a different signal into an ambient of the room after a predetermined duration of the sensor not detecting occupancy or door movement.

6. The receptacle of claim 1, further comprising:
a first port arranged along a surface of the receptacle in the bay;
a second port arranged along a surface of the receptacle not arranged in the bay; and
an air duct extending between the first port and second port.

7. A receptacle for storing a portable disinfection apparatus, comprising:
a bay for receiving the portable disinfection apparatus;
a first port arranged along a surface of the receptacle in the bay;
a second port arranged along a surface of the receptacle not arranged in the bay;
an air duct extending between the first port and second port;
a sensor, wherein the sensor is an occupancy sensor or a door movement sensor; and
a wireless transmitter communicably coupled to the sensor for sending:
a sensing signal into an ambient of a room in which the receptacle is arranged upon the sensor detecting occupancy or door movement; and
a non-sensing signal into an ambient of the room after a predetermined duration of the sensor not detecting occupancy or door movement.

8. The receptacle of claim 7, further comprising:
a power cord, a power socket, or a battery; and
an electrical contact arranged along an exterior surface of the bay for mating with a power charging contact of the portable disinfection apparatus.

9. The receptacle of claim 8, further comprising a battery charger device configured to regulate the amount of current discharged through the electrical contact.

10. The receptacle of claim 7, wherein the bay is void of electrical contacts for charging the portable disinfection apparatus.

11. The receptacle of claim 7, wherein the receptacle is smaller than the portable disinfection apparatus.

12. The receptacle of claim 7, wherein the sensor is pivotally secured to the receptacle.

13. The receptacle of claim 7, wherein the sensor is detachable from the receptacle, and wherein the receptacle comprises multiple locations at which to readily attach the sensor.

14. A receptacle for storing a portable disinfection apparatus, comprising:
a bay for receiving the portable disinfection apparatus;
a first sensor, wherein the first sensor is an occupancy sensor or a door movement sensor;
a wireless transmitter communicably coupled to the first sensor for sending a signal into an ambient of a room in which the receptacle is arranged upon the first sensor detecting occupancy or door movement;
a second sensor for detecting if the bay is occupied by the portable disinfection apparatus; and
a controller for:
inhibiting activation of the first sensor when the second sensor detects the bay is occupied; and
activating the first sensor subsequent to the second sensor detecting the bay is not occupied.

15. The receptacle of claim 14, further comprising:
a power cord, a power socket, or a battery; and
an electrical contact arranged along an exterior surface of the bay for mating with a power charging contact of the portable disinfection apparatus.

16. The receptacle of claim 15, further comprising a battery charger device configured to regulate the amount of current discharged through the electrical contact.

17. The receptacle of claim 14, wherein the bay is void of electrical contacts for charging the portable disinfection apparatus.

18. The receptacle of claim 14, wherein the sensor is pivotally secured to the receptacle.

19. The receptacle of claim 14, wherein the sensor is detachable from the receptacle, and wherein the receptacle comprises multiple locations at which to readily reattach the sensor.

20. The receptacle of claim 14, wherein the wireless transmitter is further for sending a different signal into an ambient of the room after a predetermined duration of the sensor not detecting occupancy or door movement.
